SEQUENCE LISTING (17 sequences)

SEQ ID NO: 1 - protein, 138 aa, Mus musculus
MEFGLSWLFLVAILKGVQCEVQLQQSGTVLARPGASVKMSCKASGYSFTIYWIHWVKQRPGQGLEWIATIYPGNSDIIYNQKFKGKAKLTAVTSASTAYMELSSLTNEASAVYYCTRQGYDYYAMDYWGQGTSVTVSS

SEQ ID NO: 2 - protein, 129 aa, Mus musculus
MDMRVPAQLLGLLLLWLPGARCDVQITQSPSYLAASPGETIIINCRASKSISKYLAWYQEKPGKTNKLLIYSGSTLQSGIPSRFSGSGSGTDFTLTISSLEPQDFAMYYCQQHNEYPWTFGGGTKLEIK

SEQ ID NO: 3 - DNA, 414 bp, Mus musculus
atggagttcggcctgagctggctgttcctggtggctattcttaagggtgtccagtgtgaggttcagctccagcagtctgggactgtgctggcaaggcctggggcttccgtgaagatgtcctgcaaggcttctggctacagttttaccatctactggatacactgggtaaaacagaggcctggacagggtctagaatggattgctactatttatcctgggaatagtgatattatttacaaccagaagttcaagggcaaggccaaactgactgcggtcacatccgccagcactgcctacatggagctcagcagcctgacaaatgaggcctctgcggtctattactgtacaagacaggggtacgattattatgctatggactattggggtcaaggaacctcagtcaccgtctcctca

SEQ ID NO: 4 - DNA, 386 bp, Mus musculus
atggacatgcgtgtgcccgctcaactcctgggcctgctgctgctctggctcccaggtgcgcgctgtgatgtccagataacccagtctccatcttatcttgctgcatctcctggagaaaccatcattattaattgcagggcaagtaagagcattagcaaatatttagcctggtatcaagagaaacctgggaaaactaataagcttcttatctactctggatccactttgcaatctggaattccatcaaggttcagtgcagtggatctggtacagatttcactctcaccatcagtagcctggagcctcaagattttgcaatgtattactgtcaacagcataatgaatacccgtggacgttcggtggaggcaccaagctggaaatcaaa

SEQ ID NO: 5 - protein, 330 aa, Homo sapiens [sequence omitted]

SEQ ID NO: 6 - protein, 106 aa, Homo sapiens
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSRPSQRASTGES

SEQ ID NO: 7 - DNA, 990 bp, Homo sapiens
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 8 - DNA, 320 bp, Homo sapiens
cggacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagtcgcccgtcacaaagagcttcaacaggggagagtgt

SEQ ID NO: 9 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - DNA, 977 bp, Homo sapiens
gcttccaccaagggcccatccgtcttccccctggcgccctgctccaggagcacctccgagagcacagcgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaatgatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 11 - protein, 108 aa, Mus musculus
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKR

SEQ ID NO: 12 - DNA, 324 bp, Mus musculus
aacattgtaatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggaccaagctggaaataaaacgg

SEQ ID NO: 13 - protein, 130 aa, Mus musculus
MDMRVPAQLLGLLLLWLPGARCDVQITQSPSYLAASPGETIIINCRASKSISKYLAWYQEKPGKTNKLLIYSGSTLQSGIPSRFSGSGSGTDFTLTISSLEPQDFAMYYCQQHNEYPWTFGGGTKLEIKR

SEQ ID NO: 14 - protein, 106 aa, Homo sapiens
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSRPSQRASTGEC

SEQ ID NO: 15 - protein, 129 aa, Mus musculus
MDMRVPAQLLGLLLLWLPGARCDVQITQSPSYLAASPGETIIINCRASKSISKYLAWYQEKPGKTNKLLIYSGSTLQSGIPSRFSGSGSGTDFTLTISSLEPQDFAMYYCQQHNEYPWTFGGGTKLQIK

SEQ ID NO: 16 - protein, 108 aa, Mus musculus
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLQIKR

SEQ ID NO: 17 - protein, 130 aa, Mus musculus
MDMRVPAQLLGLLLLWLPGARCDVQITQSPSYLAASPGETIIINCRASKSISKYLAWYQEKPGKTNKLLIYSGSTLQSGIPSRFSGSGSGTDFTLTISSLEPQDFAMYYCQQHNEYPWTFGGGTKLQIKR

(19) United States
(12) Patent Application Publication
(10) Pub. No.: US 2013/0216476 A1
(43) Pub. Date: Aug. 22, 2013

(54) USE OF AN ANTI-CD71 ANTIBODY FOR PREPARING A MEDICAMENT

(71) Applicant: Laurence Boumsell, Paris (FR)

(72) Inventor: Laurence Boumsell, Paris (FR)

(73) Assignee: LFB BIOTECHNOLOGIES, Courtaboeuf Cedex (FR)

(21) Appl. No.: 13/696,097

(22) PCT Filed: May 3, 2011
(86) PCT No.: PCT/FR2011/051005

§ 371 Date: May 9, 2013

(30) Foreign Application Priority Data
May 3, 2010 (FR) 1053426

(51) Int. Cl. A61K 39/395; A61K 31/69; G01N 33/574; A61K 47/48

(52) U.S. Cl. CPC A61K 39/39558; A61K 47/4863; A61K 31/69; G01N 33/57492
USPC 424/1.49; 424/144.1; 424/135.1; 424/133.1; 424/178.1; 424/183.1; 435/7.23

(57) ABSTRACT

The use of an anti-CD71 monoclonal antibody or a fragment of an abovementioned antibody capable of binding to the CD71 antigen for the preparation of a drug intended for the prevention or treatment of myelomas.

The present invention relates to the use of anti-CD71 antibodies or fragments of anti-CD71 antibodies for the preparation of a drug intended for the prevention or treatment of cancers.

CD71 is a type II glycoprotein which exists in the form of a 180 kDa homodimer. CD71 is a receptor of transferrin, a protein responsible for transporting iron from the intestine to the hepatic reserves and the reticulocytes.

Ferrotransferrin, a transferrin combined with Fe3+, is bound to the CD71 at neutral pH, and is internalized into the endosomal compartment at approximately pH 5. The Fe3+ ions are then transported to the cytoplasm by an unknown mechanism. After the release of the Fe3+ ions, apotransferrin, a transferrin without Fe3+, remains bound to CD71 at pH 5 and returns to the cell membrane at approximately pH 7.4. Under neutral pH conditions, the apotransferrin no longer has any affinity for CD71. The dissociation of the complex formed by the apotransferrin and the CD71 allows another transport cycle to commence.

CD71 plays a significant role in cell proliferation, as it controls iron consumption, an essential process in several metabolic routes. This control is implemented by the binding and the endocytosis of the transferrin by CD71. The expression of CD71 is post-transcriptionally regulated by the stability of the CD71 RNA, and the intracellular level of iron. In the presence of an iron deficiency signal, IRP-1 and IRP-2 (iron response protein) bind to specific sequences, denoted IREs (iron response elements), situated in the 5′UTR region of the CD71 RNA. This binding stabilizes the CD71 RNA. On the other hand, when the iron level is sufficiently high, the affinity of the IRP-1 and IRP-2 for the IREs is reduced and the CD71 RNA becomes more susceptible to degradation by the RNases.

Myeloma, also known as multiple myeloma, or Kahler's disease, is a cancer of the plasmocytes. The latter are produced in the spleen and migrate into the bone marrow. The plasmocytes are specialized B lymphocytes which normally produce antibodies. The normal plasmocytes are characterized by an inability to proliferate. Nevertheless, when these cells become malignant, they acquire a great ability to proliferate and all secrete the same type of immunoglobulin. In most cases, this immunoglobulin is IgG or IgA, but it can be also IgD, IgE or IgM. As a result, the blood or urine of patients suffering from a myeloma often contain a very large quantity of a single type of antibody called paraprotein. When a blood or urine sample from such a patient is analyzed by electrophoresis, these paraproteins often form a monoclonal peak. Sometimes, instead of secreting a complete immunoglobulin, the tumour plasmocytes secrete an incomplete part of immunoglobulin referred to as “Kappa or Lambda light chains”. In the latter case, there is little or no monoclonal peak in the blood, however there is severe hypogammaglobulaemia and proteinuria in the urine, produced by Kappa or Lambda chains, known as “BENCE JONES”.

In France, the incidence of this disease is approximately 4000 new cases annually. The average survival is 5 years, but depends on the stage of the disease.

The myelomas can be divided into 3 stages according to the Durie and Salmon classification established in 1986. This classification is based on the quantity of paraprotein produced, the severity of the bone lesion, the blood calcium level and the insufficiency of medullary production (haemoglobin). Stage I corresponds to a myeloma of low tumour mass, whereas stage III is a myeloma of high tumour mass. Any impairment of the renal function adds a sub-classification, in which stage A exhibits no renal impairment and stage B is characterized by renal insufficiency.

In the last few years, new classification systems based on the detection of genetic markers allow a more precise prognosis about the survival of a patient. For example, the deletion of chromosome 13q (Fonseca et al., Blood 2003; 101: 4569-4575) or of the 17p13 locus (locus for a tumour-suppressor gene) (Avet-Loiseau et al., Blood 2007; 109: 3489-3495) in the plasmocytes can mean a very poor prognosis.

The standard treatment of myeloma is based mainly on treatment by chemotherapy, which has the objective of controlling the proliferation of the tumour cells. At present, chemotherapeutic agents used in the treatment of myelomas are alkylating agents, such as melphalan or cyclophosphamide, often combined with cortisone drugs, such as prednisone, or proteasome inhibitors, such as bortezomib (Velcade®), or anti-angiogenic agents, such as thalidomide. Although these drugs have contributed to a certain prolongation of patient survival, their side-effects are also considerable. Furthermore, these drugs are rarely able to provide a permanent cure.

Another approach to myeloma treatment makes use of the autologous stem cell transplant technique. These techniques consist of the use of healthy stem cells originating from the peripheral blood or bone marrow of the patient himself. The stem cells thus recovered can be frozen until the time of use and then thawed and reinjected or retransplanted into the patient's body by intravenous route. Approximately two weeks after the injection, the stem cells can begin to produce new blood cells and regenerate the bone marrow. During the last 10 years, autologous stem cell transplant has become a treatment of choice. This is because, if the patients are able to cope with this treatment, this method makes it possible to give these patients a better survival rate. However, given that an autologous stem cell transplant is often combined with a chemotherapy before the transplant, at a high dose, this method can lead to very serious side-effects, such as a significant reduction in white blood cells.

As a result, it is necessary to develop novel drugs or novel approaches in order to increase the survival rate of patients suffering from myelomas.

A first aspect of the invention has the objective of proposing a novel drug intended for the prevention or treatment of myelomas.

Another aspect of the invention is aimed at providing a method of diagnosing myelomas in vitro.

The present invention relates to the use of an anti-CD71 monoclonal antibody or of a fragment of an abovementioned antibody capable of binding to the CD71 antigen, said antibody or said fragment of the abovementioned antibody comprising:
at least the variable region of a heavy chain comprising or constituted by the amino acid sequence SEQ ID NO: 1, and
at least the variable region of a light chain comprising or constituted by an amino acid sequence chosen from SEQ ID NO: 2, SEQ ID NO: 11, SEQ ID NO: 13, SEQ ID NO: 15, SEQ ID NO: 16 or SEQ ID NO: 17,

for the preparation of a drug intended for the prevention or treatment of myelomas.

The present invention is based on an unexpected finding made by the Inventors, during a study of the level of expression of CD71 on myeloma tumour cell lines. In fact, CD71 is overexpressed on the plasmocytes which become malignant. The invention shows both in vitro and in vivo that the blocking of CD71 by an anti-CD71 antibody makes it possible to inhibit the development of myelomas and prolong the survival of mice having a myeloma.

Several mechanisms can be involved in the inhibition of the myeloma development by an anti-CD71 antibody.

The first mechanism is the inhibition of myeloma cell proliferation by blocking the supply of iron to the tumour cells by means of the anti-CD71 antibody.

An anti-CD71 antibody can also trigger or activate an immune response, such as the production of cytokines, or the induction of apoptosis in the tumour cells, which makes it possible to destroy the cancer cells.

The myeloma cells can also be eliminated specifically by effector cells by the ADCC (antibody-dependent cell-mediated cytotoxicity) or CDC (complement-dependent cytotoxicity) process.

In the invention, the term “antibody” refers to an immunoglobulin, a multimeric protein constituted by 4 chains participating in the acquired immune response.

The immunoglobulins are well known to a person skilled in the art and are constituted by an assembly of two dimers each constituted by a heavy chain and a light chain. The multimeric complex is assembled by the binding of a light chain and a heavy chain by means of a disulphide bridge between two cysteines, the two heavy chains themselves also being bound to each other by two disulphide bridges.

Each of the heavy chains and light chains is constituted by a constant region and a variable region. The assembly of the chains which compose an antibody makes it possible to define a characteristic three-dimensional Y-shaped structure, where
the base of the Y corresponds to the constant region Fc that is recognized by complement and the Fc receptors, and the base of the Y corresponds to the constant region Fc which is recognized by the complement and the Fc receptors, and
the end of the arms of the Y correspond to the respective assembly of the variable regions of the light chain and the heavy chain.

More precisely, each light chain is constituted by a variable region (VL) and a constant region (CL). Each heavy chain is constituted by a variable region (VH) and a constant region constituted by three constant domains CH1, CH2 and CH3. The association of the two fields CH2 and CH3 compose the Fc domain.

The variable region of the light chain is constituted by three regions determining the recognition of the antigen (CDR) surrounded by four framework regions (FR). The three-dimensional folding of the variable region is such that the 3 CDRs are exposed on the same side of the protein and allow the formation of a specific structure recognizing a particular antigen.

The expression “the ability to bind to the CD71 antigen” means the ability of an antibody to recognize and specifically bind to an antigen. This specificity is conferred by the variable region of the light chain or of the heavy chain.

The ability of an antibody of recognize and specifically bind to an antigen can be tested by techniques such as ELISA, indirect immunofluorescence, or western-blot.

“A fragment of an abovementioned antibody capable of binding to the CD71 antigen” can be a Fab, F(ab)′2, scFV fragment, ScFv dimer or also a diabody, a triabody or a tetrabody.

A Fab fragment is constituted by a light chain comprising a constant region of a light chain of a human immunoglobulin and a variable region of a murine immunoglobulin, and by a heavy chain comprising a constant region of a heavy chain of a human immunoglobulin and by a variable region of a murine immunoglobulin.

A F(ab)′ 2 fragment is constituted by the combination of two Fab fragments described above by means of a disulphide bridge.

An scFv fragment is constituted by the combination of a heavy chain variable fragment and a light chain variable fragment of an immunoglobulin.

An ScFv dimer is constituted by two covalently bound ScFv fragments, each ScFv being itself constituted by a heavy chain variable fragment and a light chain variable fragment.

A diabody is constituted by the non-covalent combination of two polypeptides themselves containing two variable chains of the same kind (VL-VL and VH-VH) which can be of the same specificity or of different specificities.

A triabody or a tetrabody can be constituted in the same way.

In an embodiment, the invention relates to the use of an anti-CD71 monoclonal antibody or fragment of an abovementioned antibody capable of binding to the CD71 antigen, said antibody or said fragment of the abovementioned antibody comprising:
at least the variable region of a heavy chain comprising or constituted by an amino acid sequence encoded by a nucleic sequence SEQ ID NO: 3, and
at least the variable region of a light chain comprising or constituted by an amino acid sequence encoded by a nucleic sequence chosen from SEQ ID NO: 4 and SEQ ID NO: 12, for the preparation of a drug intended for the prevention or treatment of myelomas.

In the present invention, the nucleic sequence SEQ ID NO: 3 encodes the amino acid sequence SEQ ID NO: 1. The nucleic sequences SEQ ID NO: 4 and SEQ ID NO: 12 encode the amino acid sequences SEQ ID NO: 2 and SEQ ID NO: 11 respectively.

Advantageously, the invention relates to the use of an anti-CD71 antibody as defined above, where the constant regions of said light chains and heavy chains are constant regions of a human antibody.

Said human antibody can be an antibody of IgG, IgA, IgD, IgE, IgM type.

This embodiment of the invention makes it possible to reduce the immunogenicity of the antibody in humans and thereby improve its effectiveness during its therapeutic or prophylactic administration to humans.

The constant region of each of the heavy chains of the antibody according to the invention is in particular the constant region of human IgG1, IgG2, IgG3, IgG4, IgM, IgD, IgE, IgA 1 or IgA2.

Advantageously, the constant region of each of the heavy chains of the antibody according to the invention is the constant region of a human immunoglobulin of G1 type, as such an antibody exhibits an ability to produce ADCC activity in the greatest number of (human) individuals.

Advantageously, the constant region of each of the light chains of the antibody according to the invention is the constant region of the κ (kappa) or λ (lambda) chain.

In a particular embodiment, the invention relates to the use of an anti-CD71 antibody defined previously, in which:
at least the constant region of a light chain comprises or is constituted by the amino acid sequence chosen from SEQ ID NO: 6 and SEQ ID NO: 14
at least the constant region of a heavy chain comprises or is constituted by the amino acid sequence chosen from SEQ ID NO: 5 or SEQ ID NO: 9.

In another embodiment, the invention relates to the use of an anti-CD71 antibody as defined above, in which:
at least the constant region of a light chain comprises or is constituted by the amino acid sequence encoded by the nucleic sequence SEQ ID NO: 8,
at least the constant region of a heavy chain comprises or is constituted by the amino acid sequence encoded by a nucleic sequence chosen from SEQ ID NO: 7 or SEQ ID NO: 10.

In the present invention, the nucleic sequence SEQ ID NO: 8 encodes the amino acid sequence SEQ ID NO: 6. The nucleic sequences SEQ ID NO: 7 and SEQ ID NO: 10 encode the amino acid sequences SEQ ID NO: 5 and SEQ ID NO: 9 respectively.

In a particular embodiment, the anti-CD71 antibody for the implementation of the invention can be an antibody where:
the variable region of each of its light chains comprises or is constituted by the amino acid sequence SEQ ID NO: 2, and
the variable region of each of its heavy chains comprises or is constituted by the amino acid sequence SEQ ID NO: 1, and
the constant region of each of its light chains comprises or is constituted by the amino acid sequence SEQ ID NO: 14, and
the constant region of each of its heavy chains comprises or is constituted by the amino acid sequence chosen from SEQ ID NO: 5 or SEQ ID NO: 9.

Advantageously, the anti-CD71 antibody for the implementation of the invention is produced by the hybridoma BA120g, deposited at the CNCM under number CNCM I-3449 on 14 Jun. 2005.

An anti-CD71 monoclonal antibody for the implementation of the present invention can be produced by conventional methods known to a person skilled in the art, for example, the construction of a DNA vector which expresses:
the heavy chain, or
a fragment of the heavy chain, or
the light chain, or
a fragment of the light chain, or
the heavy chain and the light chain, or
a fragment of the heavy chain and a fragment of the light chain.

These vectors are for example plasmids, cosmids, yeast artificial chromosomes, bacterial artificial chromosomes and artificial chromosomes derived from the bacteriophage P1, and vectors derived from viruses.

In an embodiment of the invention, a fragment of an anti-CD71 antibody put to the use described above can be Fab, F(ab)′2, scFV, ScFv dimer or also a diabody, a triabody or a tetrabody.

These fragments can be synthetic, recombinant fragments, or produced by enzymatic cleavage according to the methods known to a person skilled in the art. These fragments can also be produced by introducing one or more stop codons into an anti-CD71 antibody encoding gene. For example, when a stop codon is introduced after the CH1 domain of the gene encoding the heavy chain, the chain thus produced is a heavy chain capable of forming a Fab fragment with a light chain. On the other hand, if a stop codon is added after the hinge region situated downstream of the CH1 domain of a heavy chain, this truncated chain can constitute a F(ab)′2 fragment with a light chain.

In another particular embodiment of the invention, the anti-CD71 antibody implemented as described above is coupled with a bioactive molecule.

This bioactive molecule can be a radio-isotope, for example a gamma radiation emitter, a beta radiation emitter or an alpha radiation emitter.

Advantageously, a radio-isotope coupled to the anti-CD71 antibody or a fragment of the anti-CD71 antibody is a radio-isotope capable of being used for a therapeutic or diagnostic purpose, such as Actinium 225, Actinium 227, Arsenic 72, Astatine 211 (PET (Positon Emission Topography)), Bismuth 212 or 213, Bromine 75 (PET), Bromine 77, Cobalt 55 (PET), Copper 61 (PET), Copper 64 (PET and treatment), Copper 67(PET), Iodine 123 (PET), Iodine 131, Lutetium 177 (PET and treatment), Osmium 194, Radon 223, Rhenium 186, Ruthenium 105, Terbium 149, Thallium 228 and 229, Yttrium 90 and 91.

These radio-isotopes can be bound to the antibodies directly or by means of a chelator, such as DTA (9,10-dithioanthracene), or DTPA (diethylene triamine penta acid).

A bioactive molecule can be also a non-radioactive metal.

Said bioactive molecule can also be:
a toxin, for example ricin, abrin, diphtheria toxin, Phytolacca antiviral protein, or a functional fragment of a toxin;
a nucleic acid such as an anti-sense RNA, an siRNA, or an miRNA;
a cytotoxic agent, for example mitomycin C, methotrexate, adriamycin, doxorubicin, daunorubicin, vinblastine, bleomycin, taxol, taxotere, navelbine;
an enzyme such as an RNase;
a galenic vector, for example liposomes, nanoparticles, polymers, cationic emulsions, anionic emulsions, neutral emulsions, or dendritomes;
another antibody or a fragment of another antibody which recognizes an antigen other than CD71;
biotin, avidin or streptavidin.

An abovementioned bioactive molecule can be bound to the antibody directly or by means of a linker.

A particular aspect of the invention relates to the use
of an anti-CD71 monoclonal antibody or of a fragment of an above-mentioned antibody capable of binding to the CD71 antigen, said antibody or said fragment of the abovementioned antibody comprising:
   at least the variable region of a heavy chain comprising or constituted by the amino acid sequence SEQ ID NO: 1, and
   at least the variable region of a light chain comprising or constituted by an amino acid sequence chosen from SEQ ID NO: 2, SEQ ID NO: 11, SEQ ID NO: 13, SEQ ID NO: 15, SEQ ID NO: 16 or SEQ ID NO: 17, and
effector cells,
   for the preparation of a drug intended for the prevention or treatment of myelomas.

Effector cells are used simultaneously with the anti-CD71 antibody for preparing a drug intended for the prevention or treatment of myelomas.

These effector cells are cells capable of expressing Fc domain receptors, such as human

T lymphocytes, human NK lymphocytes, human monocytes, human granulocytes, or human macrophages. The interaction of the Fc receptor on the effector cells with the Fc domain of the anti-CD71 antibody makes it possible to induce an ADCC activity directed at the target cells, i.e. the cells recognized by the specific antibody, and subsequently eliminate them.

Effector cells can be autologous or allogeneic cells. Advantageously, effector cells are cells originating from a patient needing myeloma treatment.

Effector cells are separated from the peripheral blood taken from a donor or a patient according to the conventional methods well known to a person skilled in the art (Boyum et al., Scand. J. Clin. Lab. Invest. Suppl. 1976. 5:9-15).

In an advantageous embodiment, the invention relates to the use of an anti-CD71 antibody described above and effector cells for preparing a drug intended for the prevention or treatment of myelomas, in which the constant regions of the light chains and heavy chains of said antibodies are constant regions of a human antibody.

In a more advantageous embodiment, the invention relates to the use of an anti-CD71 antibody described above and effector cells for preparing a drug intended for the prevention or treatment of myelomas, in which:
at least the constant region of a light chain comprises or is constituted by the amino acid sequence SEQ ID NO: 14,
at least the constant region of a heavy chain comprises or is constituted by the amino acid sequence chosen from SEQ ID NO: 5 or SEQ ID NO: 9.

In a more advantageous embodiment, the invention relates to the use of an anti-CD71 antibody described above and effector cells for preparing a drug intended for the prevention or treatment of myelomas, in which:
the variable region of each of its light chains comprises or is constituted by the amino acid sequence SEQ ID NO: 2, and
the variable region of each of its heavy chains comprises or is constituted by the amino acid sequence SEQ ID NO: 1, and
the constant region of each of its light chains comprises or is constituted by the amino acid sequence SEQ ID NO: 14, and
the constant region of each of its heavy chains comprises or is constituted by the amino acid sequence chosen from SEQ ID NO: 5 or SEQ ID NO: 9.

More advantageously, the invention relates to the use of the effector cells and of an anti-CD71 antibody produced by the hybridoma BA120g, deposited at the CNCM on 14 Jun. 2005 under number CNCM I-3449, for preparing a drug intended for the prevention or treatment of myelomas.

In another advantageous embodiment of the invention, the invention relates to the use of an anti-CD71 antibody described above and effector cells for preparing a drug intended for the prevention or treatment of myelomas, in which the antibody is coupled to a bioactive molecule chosen from:
radio-isotopes,
non-radioactive metals,
toxins chosen from ricin, abrin, diphtheria toxin,
nucleic acids chosen from the anti-sense RNAs,
cytotoxic agents chosen from mitomycin C, methotrexate, adriamycin,
enzymes such as the RNases,
biotin, avidin or streptavidin.

In a particular embodiment, the anti-CD71 antibody described above is intended for the prevention or treatment of a myeloma chosen from stage I myeloma (low tumour mass), stage II myeloma (intermediate tumour mass), or stage III myeloma (high tumour mass).

The classification of myelomas is based on the criteria established by Doctor Durie and Doctor Salmon in 1986. It is a classification based on the quantity of paraprotein produced, severity of bone lesion, blood calcium level and insufficiency of medullary production (haemoglobin).

Stage I corresponds to a myeloma of low tumour mass, in which all the following criteria are present:
Haemoglobin>100 g/1
Calcaemia<120 mg/l (3 mmol/l)
Absence of bone lesion or a isolated bone plasmacytoma
Low monoclonal Ig rate:
   IgG<50 g/l
   IgA<30 g/l
   Bence Jones proteinuria<4 g/24 H.

Stage II does not correspond to the definition either of stage I, or stage III.

Stage III is a myeloma of high tumour mass, which exhibits at least one of the following criteria:
Haemoglobin<8.5 g/dl
Calcaemia>120 mg/l (>3 mmol/l)
Multiple bone lesions (>3)
High monoclonal Ig rate:
   IgG>70 g/l
   IgA>50 g/l
   Bence Jones proteinuria>12 g/24 H

A subject of another aspect of the present invention is to propose an anti-CD71 antibody for the treatment of myelomas.

In an embodiment, the invention relates to an anti-CD71 monoclonal antibody or a fragment of an abovementioned antibody capable of binding to the CD71 antigen, said antibody or said fragment of the abovementioned antibody comprising

at least the variable region of a heavy chain comprising or constituted by the amino acid sequence SEQ ID NO: 1, and

at least the variable region of a light chain comprising or constituted by an amino acid sequence chosen from SEQ ID NO: 2, SEQ ID NO: 11, SEQ ID NO: 13, SEQ ID NO: 15, SEQ ID NO: 16 or SEQ ID NO: 17,

for the treatment of myelomas.

In another embodiment, the invention relates to an anti-CD71 monoclonal antibody or a fragment of an abovementioned antibody capable of binding to the CD71 antigen, said antibody or said fragment of the abovementioned antibody comprising:
at least the variable region of a heavy chain comprising or constituted by an amino acid sequence encoded by a nucleic sequence SEQ ID NO: 3, and
at least the variable region of a light chain comprising or constituted by an amino acid sequence encoded by a nucleic sequence chosen from SEQ ID NO: 4 and SEQ ID NO: 12, for the treatment of myelomas.

In an advantageous embodiment, the invention relates to an anti-CD71 antibody for the treatment of myelomas, in which the constant regions of said light chains and heavy chains are constant regions of a human antibody.

In a more advantageous embodiment, the invention relates to an anti-CD71 antibody, in which:
at least the constant region of a light chain comprises or is constituted by the amino acid sequence SEQ ID NO: 14,
at least the constant region of a heavy chain comprises or is constituted by the amino acid sequence chosen from SEQ ID NO: 5 or SEQ ID NO: 9, for the treatment of myelomas.

In another more advantageous embodiment, the invention relates to an anti-CD71 antibody, in which:
at least the constant region of a light chain comprises or is constituted by the amino acid sequence encoded by the nucleic sequence SEQ ID NO: 8,
at least the constant region of a heavy chain comprises or is constituted by the amino acid sequence encoded by a nucleic sequence chosen from SEQ ID NO: 7 or SEQ ID NO: 10, for the treatment of myelomas.

In a particularly advantageous embodiment, the invention relates to an anti-CD71 antibody, in which:
the variable region of each of its light chains comprises or is constituted by the amino acid sequence SEQ ID NO: 2, and
the variable region of each of its heavy chains comprises or is constituted by the amino acid sequence SEQ ID NO: 1, and
the constant region of each of its light chains comprises or is constituted by the amino acid sequence SEQ ID NO: 14, and
the constant region of each of its heavy chains comprises or is constituted by the amino acid sequence chosen from SEQ ID NO: 5 or SEQ ID NO: 9, for the treatment of myelomas.

In an advantageous embodiment, the antibody utilized for the treatment of myeloma is produced from a hybridoma BA120g, deposited at the CNCM on 14 Jun. 2005 under the number CNCM I-3449.

The anti-CD71 antibody described above and intended for the treatment of myelomas can be combined with pharmaceutically acceptable excipients or vehicles.

The anti-CD71 antibody described above and intended for the treatment of myelomas can be administered by intravenous route, for example by injection or perfusion.

In a particular embodiment, an anti-CD71 antibody described above for the treatment of myelomas, is coupled to a bioactive molecule chosen from:
radio-isotopes,
non-radioactive metals,
toxins chosen from ricin, abrin, diphtheria toxin,
nucleic acids chosen from the anti-sense RNAs,
cytotoxic agents chosen from mitomycin C, methotrexate, adriamycin,
enzymes such as the RNases,
biotin, avidin or streptavidin

Another aspect of the invention relates to a product containing:
at least one anti-CD71 monoclonal antibody or a fragment of the above-mentioned antibody capable of binding to the CD71 antigen, said antibody or said fragment of the abovementioned antibody comprising:
   at least the variable region of a heavy chain comprising or constituted by the amino acid sequence SEQ ID NO: 1, and
   at least the variable region of a light chain comprising or constituted by an amino acid sequence chosen from SEQ ID NO: 2 and SEQ ID NO: 11,
an anticancer agent chosen from Velcade®, Melphalan®, Prednisone® as combination product, for use simultaneously, separately or spread over time in tumour therapy.

In a particular embodiment, the invention relates to a product containing:
at least one anti-CD71 monoclonal antibody or a fragment of the above-mentioned antibody capable of binding to the CD71 antigen, said antibody or said fragment of the abovementioned antibody comprising:
   at least the variable region of a heavy chain comprising or constituted by the amino acid sequence SEQ ID NO: 1, and
   at least the variable region of a light chain comprising or constituted by an amino acid sequence chosen from SEQ ID NO: 2, SEQ ID NO: 11, SEQ ID NO: 13, SEQ ID NO: 15, SEQ ID NO: 16 or SEQ ID NO: 17, and
   the constant regions of the light chains and heavy chains originating from a human antibody,
an anticancer agent chosen from Velcade®, Melphalan®, Prednisone®,
   as combination product, for use simultaneously, separately or spread over time in tumour therapy.

In a particular embodiment, the invention relates to a product containing:
at least one anti-CD71 monoclonal antibody or a fragment of the above-mentioned antibody capable of binding to the CD71 antigen, said antibody or said fragment of the abovementioned antibody comprising:
   at least the variable region of a heavy chain comprising or constituted by the amino acid sequence SEQ ID NO: 1, and
   at least the variable region of a light chain comprising or constituted by an amino acid sequence chosen from SEQ ID NO: 2, SEQ ID NO: 11, SEQ ID NO: 13, SEQ ID NO: 15, SEQ ID NO: 16 or SEQ ID NO: 17, and
   at least the constant region of a light chain comprises or is constituted by the amino acid sequence SEQ ID NO: 6 and SEQ ID NO: 14,
   at least the constant region of a heavy chain comprises or is constituted by the amino acid sequence chosen from SEQ ID NO: 5 or SEQ ID NO: 9,
an anticancer agent chosen from Velcade®, Melphalan®, Prednisone®,
   as combination product, for use simultaneously, separately or spread over time in tumour therapy.

In a particular embodiment, the invention relates to a product containing:
at least one anti-CD71 monoclonal antibody or a fragment of the above-mentioned antibody capable of binding to the CD71 antigen, said antibody or said fragment of the abovementioned antibody comprising:
   the variable region of each of its light chains comprises or is constituted by the amino acid sequence SEQ ID NO: 2, and
   the variable region of each of its heavy chains comprises or is constituted by the amino acid sequence SEQ ID NO: 1, and
   the constant region of each of its light chains comprises or is constituted by the amino acid sequence SEQ ID NO: 14, and
   the constant region of each of its heavy chains comprises or is constituted by the amino acid sequence chosen from SEQ ID NO: 5 or SEQ ID NO: 9,
an anticancer agent chosen from Velcade®, Melphalan®, Prednisone®, as combination product, for use simultaneously, separately or spread over time in tumour therapy.

In a particularly advantageous embodiment, the invention relates to a product containing:
the anti-CD71 antibody produced by a hybridoma BA120g, deposited at the CNCM on 14 Jun. 2005 under the number CNCM I-3449,
an anticancer agent chosen from Velcade®, Melphalan®, Prednisone®,
   as combination product, for use simultaneously, separately or spread over time in tumour therapy.

In a particularly advantageous embodiment, the invention relates to a product containing:
the anti-CD71 antibody produced by a hybridoma BA120g, deposited at the CNCM on 14 Jun. 2005 under the number CNCM I-3449, and
Velcade®.

In another embodiment, the invention relates to a product containing:
at least one anti-CD71 monoclonal antibody or a fragment of the above-mentioned antibody capable of binding to the CD71 antigen, said antibody or said fragment of the abovementioned antibody comprising:
   at least the variable region of a heavy chain comprising or constituted by the amino acid sequence SEQ ID NO: 1, and
   at least the variable region of a light chain comprising or constituted by an amino acid sequence chosen from SEQ ID NO: 2, SEQ ID NO: 11 and SEQ ID NO: 13, SEQ ID NO: 15, SEQ ID NO: 16 or SEQ ID NO: 17
effector cells, and
an anticancer agent chosen from Velcade®, Melphalan®, Prednisone®,

as combination product, for use simultaneously, separately or spread over time in tumour therapy.

In a particular embodiment, the invention relates to a product containing:
at least one anti-CD71 monoclonal antibody or a fragment of the above-mentioned antibody capable of binding to the CD71 antigen, said antibody or said fragment of the abovementioned antibody comprising:
   at least the variable region of a heavy chain comprising or constituted by the amino acid sequence SEQ ID NO: 1, and
   at least the variable region of a light chain comprising or constituted by an amino acid sequence chosen from SEQ ID NO: 2, SEQ ID NO: 11, SEQ ID NO: 13, SEQ ID NO: 15, SEQ ID NO: 16 or SEQ ID NO: 17, and
   the constant regions of the light chains and heavy chains originating from a human antibody,
an anticancer agent chosen from Velcade®, Melphalan®, Prednisone®, and
effector cells,

as combination product, for use simultaneously, separately or spread over time in tumour therapy.

In a particular embodiment, the invention relates to a product containing:
at least one anti-CD71 monoclonal antibody or a fragment of the above-mentioned antibody capable of binding to the CD71 antigen, said antibody or said fragment of the abovementioned antibody comprising:
   at least the variable region of a heavy chain comprising or constituted by the amino acid sequence SEQ ID NO: 1, and
   at least the variable region of a light chain comprising or constituted by an amino acid sequence chosen from SEQ ID NO: 2 and SEQ ID NO: 11, and
   at least the constant region of a light chain comprises or is constituted by the amino acid sequence SEQ ID NO: 6 or SEQ ID NO: 14,
   at least the constant region of a heavy chain comprises or is constituted by the amino acid sequence chosen from SEQ ID NO: 5 or SEQ ID NO: 9,
an anticancer agent chosen from Velcade®, Melphalan®, Prednisone®,
effector cells,
   as combination product, for use simultaneously, separately or spread over time in tumour therapy.

In a particular embodiment, the invention relates to a product containing:
at least one anti-CD71 monoclonal antibody or a fragment of the above-mentioned antibody capable of binding to the CD71 antigen, said antibody or said fragment of the abovementioned antibody comprising:
   the variable region of each of its light chains comprises or is constituted by the amino acid sequence SEQ ID NO: 2, and
   the variable region of each of its heavy chains comprises or is constituted by the amino acid sequence SEQ ID NO: 1, and
   the constant region of each of its light chains comprises or is constituted by the amino acid sequence SEQ ID NO: 14, and
   the constant region of each of its heavy chains comprises or is constituted by the amino acid sequence chosen from SEQ ID NO: 5 or SEQ ID NO: 9,
an anticancer agent chosen from Velcade®, Melphalan®, Prednisone®, and
effector cells,
   as combination product, for use simultaneously, separately or spread over time in tumour therapy.

In a particularly advantageous embodiment, the invention relates to a product containing:
the anti-CD71 antibody produced by a hybridoma BA120g, deposited at the CNCM on 14 Jun. 2005 under the number CNCM I-3449,
an anticancer agent chosen from Velcade®, Melphalan®, Prednisone®,
effector cells,
   as combination product, for use simultaneously, separately or spread over time in tumour therapy.

Another aspect of the invention has the objective of providing a method for diagnosing myelomas in vitro.

This method is based on the unexpected finding made by the Inventors that CD71 is often overexpressed on myelomatous cells. As a result, the overexpression of CD71 on the plasmocytes can mean that these cells are becoming malignant.

This method comprises:
(i) the incubation of a cell sample taken from a patient suspected of having a myeloma, with an anti-CD71 antibody or a fragment of an abovementioned antibody capable of binding to the CD71 antigen, said antibody or said fragment of the abovementioned antibody comprising:
at least the variable region of a heavy chain comprising or constituted by the amino acid sequence SEQ ID NO: 1, and
at least the variable region of a light chain comprising or constituted by an amino acid sequence chosen from SEQ ID NO: 2, SEQ ID NO: 11 or SEQ ID NO: 13, SEQ ID NO: 15, SEQ ID NO: 16 or SEQ ID NO: 17
(ii) measurement of the quantity of CD71 antigens present on the surface of cells presented in said sample,
(iii) comparison of the value obtained in the previous stage with a reference value established in a healthy cell sample.

Advantageously, the anti-CD71 antibody used in this method is an antibody in which:
the variable region of each of its light chains comprises or is constituted by the amino acid sequence SEQ ID NO: 2, and
the variable region of each of its heavy chains comprises or is constituted by the amino acid sequence SEQ ID NO: 1, and
the constant region of each of its light chains comprises or is constituted by the amino acid sequence SEQ ID NO: 14, and
the constant region of each of its heavy chains comprises or is constituted by the amino acid sequence chosen from SEQ ID NO: 5 or SEQ ID NO: 9.

More advantageously, the anti-CD71 antibody used in this method is produced by a hybridoma BA120g, deposited at the CNCM on 14 Jun. 2005 under the number CNCM I-3449.

The anti-CD71 antibody used in this method can be used directly or coupled with a marker.

When this antibody is used directly, the presence of this antibody can be detected by another detection antibody, the latter being bound to a marker, such as a radioactive agent, or an enzyme capable of catalyzing a substrate emitting radiation by fluorescence or capable of emitting radiation at a wavelength different from that of absorption, or any other marker making it possible to detect the binding of the detection antibody to the anti-CD71 antibody.

The anti-CD71 antibody utilized in this method can be coupled beforehand with a marker making it possible to detect the binding of this antibody to the plasmocytes. This marker can be a radioactive agent, or an enzyme capable of catalyzing a substrate emitting radiation by fluorescence or capable of emitting radiation at a wavelength different from that of absorption, or any other marker making it possible to detect the binding of the anti-CD71 antibody to the plasmocytes.

The invention is illustrated by the following examples and the figures. The examples below are intended to clarify the subject-matter of the invention and illustrate advantageous embodiments, but are in no event intended to restrict the scope of the invention.

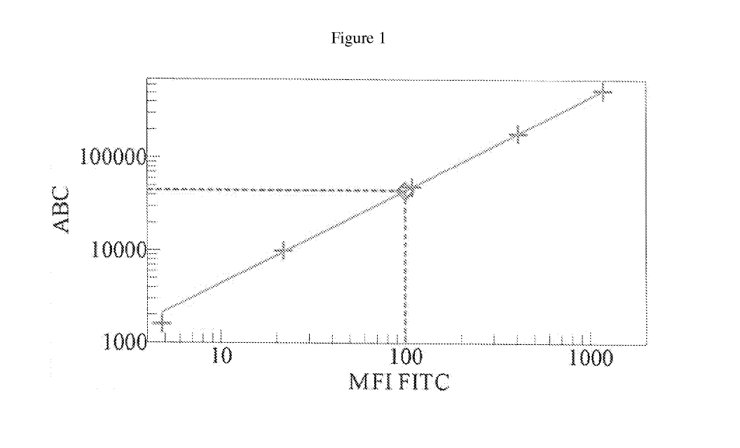

FIG. 1: FIG. 1 represents a calibration curve for measuring the ability of an antibody to bind to cells. The X-axis represents the MFI (mean fluorescence intensity) logarithm of coupled secondary antibody. The Y-axis represents the logarithm of the ability of an antibody to bind to cells. The crosses represent the data actually obtained by the measurement.

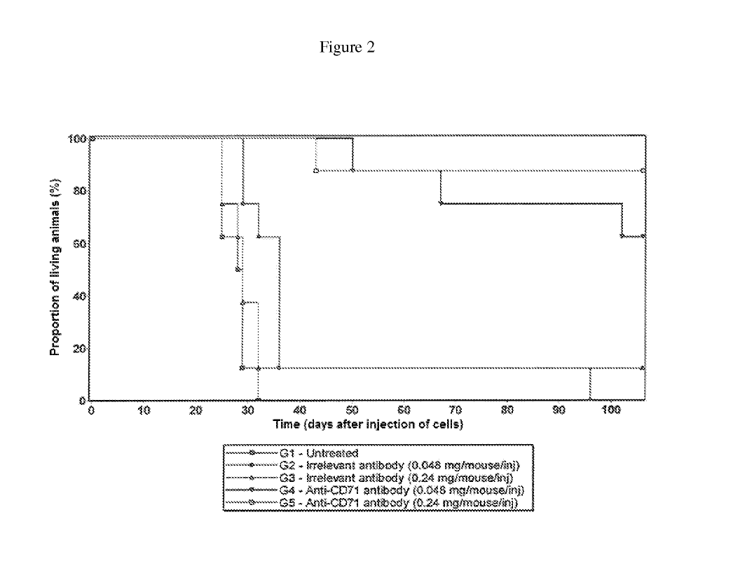

FIG. 2: FIG. 2 represents measurement of the survival of tested mice in 5 groups. Group 1 (black squares) receive nothing. Group 2 (black dots) receive an irrelevant antibody at 0.048 mg/kg/injection. Group 3 (triangle) receive an irrelevant antibody at 0.24 mg/kg/injection. Group 4 (inverted triangles) receive an anti-CD71 antibody at 0.048 mg/kg/injection. Group 5 (empty squares) receive an anti-CD71 antibody at 0.24 mg/kg/injection. The X-axis represents the number of days after the injection of tumour cells. The Y-axis represents the percentage of living mice.

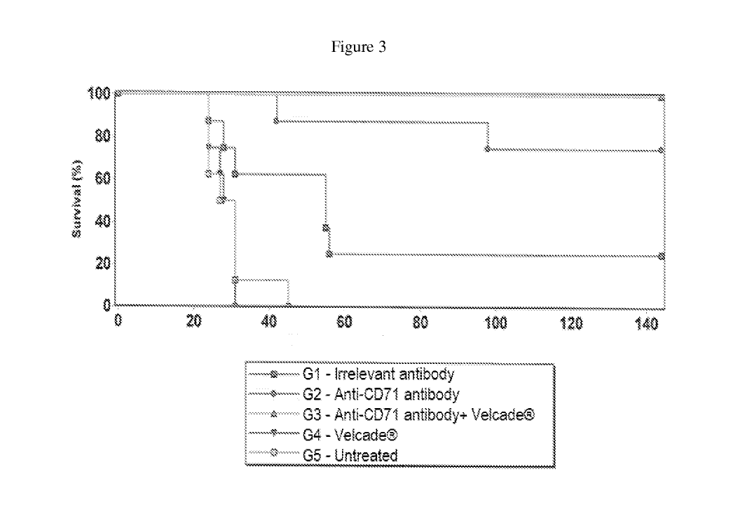

FIG. 3: FIG. 3 represents measurement of the survival of tested mice in 5 groups. Group 1 (black squares) receive an injection of 1.2 mg of an irrelevant antibody twice weekly. Group 2 (black dots) receive an injection of 1.2 mg of the anti-CD71 antibody twice weekly. Group 3 (triangle) receive an injection of 1.2 mg of the anti-CD71 antibody and an injection of 0.5 mg/kg of VELCADE® twice weekly. Group 4 (inverted triangles) receives an injection of 0.5 mg/kg of VELCADE® twice weekly. Group 5 (empty squares) receive no treatment. The X-axis represents the number of days after the injection of tumour cells. The Y-axis represents the percentage of living mice.

### RESULT

1. Detection of the CD71 Antigen by the Anti-CD71 Antibody

The detection of the CD71 antigen by the anti-CD71 antibody was measured on the ARH-77 cell line, a myeloma line.

This cell line is cultured in suspension at 37° C. in a humidified atmosphere (5% CO₂, 95% humidity). The culture medium is RPMI (Ref. #BE12-702F, Lonza, Verviers, Belgium) containing 2 mM L-glutamine and completed with 10% bovine foetal serum (Ref. #DE14-801E, Lonza).

Possible contamination of the cell culture with mycoplasmas is detected using MycoAlert® Mycoplasma Detection Kit (Ref. #LT07-318, Lonza) according to the manufacturer's instructions. This kit makes it possible to carry out a selective biochemical test which demonstrates the enzymatic activity of mycoplasmas. The cell culture supernatant is recovered and tested with a MycoAlert® kit. The test is repeated twice and the result is compared with a negative control and a positive control (Mycoalert® Assay Control Set, Ref. #L007-518, Lonza). Viable mycoplasmas are lysed and released enzymes react with the MycoAlert® kit substrate, catalyzing the conversion of ADP to ATP. Measurement of the level of ATP before and after the addition of MycoAlert® substrate makes it possible to obtain a ratio indicating the presence or absence of mycoplasmas.

The detection of anti-CD71 antibody is carried out by flow cytometry (GUAVA Personal Cell Analysis-96 (PCA-96) Systems). The data obtained is analyzed by FCS Express software (De Novo Software).

The negative control antibody is IgG1 (MAT002). The positive control antibody is an anti-CD71 antibody originating from the company MAT (MAT201). The antibody to be tested is the anti-CD71 antibody produced by the hybridoma BA120g, diluted to 10 μg/ml, 1 μg/ml, 0.1 μg/ml, 0.05 μg/ml, 0.01 μg/ml, 0.005 μg/ml. The coupled secondary antibody is an anti-mouse IgG PE antibody. The dilution and washing buffer (PBS (saline phosphate buffer) 2% FCS (fœtal calf serum)) is prepared from 49 0 ml of PBS (without Ca2+/Mg2+) and 10 ml of de-complemented FCS. PBS (Cambrex Biowhittaker BE 17-516F) is kept at ambient temperature until used. FCS (Cambrex Ref5SB0008) is kept at −20° C. until used. The Trypan blue (Sigma T8154) is kept at ambient temperature.

The control antibody concentration is 10 μg/ml.

Approximately 100,000 cells in a volume of 100 μl are first incubated with the anti-CD71 antibody in a different dilution or the control antibody respectively. The anti-CD71 antibodies are then revealed by a secondary antibody coupled to a fluorescent agent (anti-mouse IgG-PE 1/100). The results are shown in Table 1 below.

TABLE 1

| Primary antibody | CD71− % | CD71+ % | MFI |
|---|---|---|---|
| — | 94.8 | 5.2 | 6.2 |
| IgG1 controls 10 μg/ml | 96.3 | 3.7 | 4.9 |
| Anti-CD71 10 μg/ml | 5.3 | 94.7 | 32.4 |
| Anti-CD71 1 μg/ml | 10.6 | 89.4 | 28.8 |
| Anti-CD71 0.1 μg/ml | 25.2 | 74.8 | 17.6 |
| Anti-CD71 0.05 μg/ml | 34.3 | 65.7 | 12.7 |
| Anti-CD71 0.01 μg/ml | 59.9 | 40.1 | 6.6 |
| Anti-CD71 0.005 μg/ml | 70.0 | 30.0 | 5.6 |

The anti-CD71 antibody can bind specifically to the cell membrane of the ARH-77 cells compared with the IgG1 control antibodies. When the anti-CD71 antibody concentration is greater than 1 μg/ml, this antibody can recognize approximately 90% of ARH-77 cells in the sample.

2. Determination of the Number of CD71 Antigens on the Surface of ARH-77 Cells

The number of CD71 antigens on the surface of ARH-77 cells is determined by indirect immunofluorescence using a QIFIKIT kit (Dakocytomation, Trappes, France) according to the manufacturer's instructions. The immunofluorescence result is detected by flow cytometry.

The ARH-77 cells are first incubated with the anti-CD71 antibody purified from the hybridoma BA120g and an irrelevant antibody (anti-mouse IgG1) as negative control respectively. The white particles supplied in the kit are used to establish the background noise. Four populations of reference particles, having a different number of control antibodies, are used to establish the calibration curve. The antibodies binding to the cells are then revealed by a second antibody coupled with a fluorescent agent (anti-mouse IgG-FITC).

The MFI (mean fluorescence intensity) measured for the four populations of reference particles make it possible to establish a calibration curve (FIG. 1). This curve varies according to the device used. In the present experiment, the formula corresponding to this curve is as follows:

Y=A*X²+B*X+C,

in which:

A is equal to 0.013248;

B is equal to 449.282318;

C is equal to −31.588965;

X is the MFI (mean fluorescence intensity) originating from the flow cytometry reading;

Y is the total ability of an antibody to bind to a cell population.

The ability of anti-CD71 antibody to bind specifically to the ARH-77 cells (SABC) is obtained according to the following formula: SABC=ABC−BAE, in which BAE represents the background noise and ABC is the ability of the anti-CD71 antibody to bind to the ARH-77 cells. The background noise is the ability of an irrelevant antibody to bind non-specifically to the cells.

Table 2 below shows the result of the determination of the number of CD71 on ARH-77.

TABLE 2

| Antibody | MFI | ABC | BAE | SABC |
|---|---|---|---|---|
| Control IgG1 5 μg/ml | 5.2 | — | 2287 | |
| Anti-CD71 5 μg/ml | 99.6 | 44826 | — | 42539 |

3. In Vivo Test of the Anti-Tumour Effectiveness of the Anti-CD71 Antibody in Mice with Severe Combined Immunodeficiency

3.1 Effect of the Anti-CD71 Antibody on the Weight of the Mice Bearing ARH-77 Tumour Cells

Female mice with severe combined immunodeficiency (SCID), aged from 6 to 7 weeks, weighing from 16 to 20 g are obtained from Charles River (France). The mice used in the experiments are first observed for 7 days in an animal care unit free from specific pathogenic organisms. This animal care unit is authorized by the Ministry of Agriculture and the Ministry of French Research (Agreement No.A21231011). The animal experiments are implemented according to European Directive on animal experimentation and the UK Guidelines for the Welfare of Animals in Experimental Neoplasia.

The ARH-77 tumour cells expressing CD71 on the cell surface are suspended in the RPMI 1640 medium (Ref. #BE12-702F, Lonza, Verviers, Belgium) containing 2 mM L-glutamine and completed with 10% foetal calf serum (Ref. #DE14-801E, Lonza).

40 female mice with severe combined immunodeficiency (SCID) were first injected by intravenous route with a volume of 0.2 ml of the RPMI 1640 medium containing 5 million suspended ARH-77 tumour cells. The injection was carried out between 24 and 72 hours after exposure of the mice tested in this experiment to gamma radiation (1.8 Gy, ⁶⁰Co, BioMEP sarl, France).

The day of injection of the tumour cells is considered as day 0. The treatment commences on day 5.

The 40 mice are then separated into 5 groups:

Table 3 below summarizes the treatment programme for each group.

TABLE 3

| | No. of | Substance | | Treatment | Injection |
|---|---|---|---|---|---|
| Group | mice | tested | Dose | programme | volume |
| 1 | 8 | None | | | |
| 2 | 8 | Irrelevant | 0.048 mg/ | Every 3 days | 200 μL/mouse |
| | | antibody | mouse/inj | with a total | |
| | | | | of 4 | |
| | | | | injections | |
| 3 | 8 | Irrelevant | 0.24 mg/ | Every 3 days | 200 μL/mouse |
| | | antibody | mouse/inj | with a total | |
| | | | | of 4 | |
| | | | | injections | |
| 4 | 8 | Anti- | 0.048 mg/ | Every 3 days | 200 μL/mouse |
| | | CD71 | mouse/inj | with a total | |
| | | antibodies | | of 4 | |
| | | | | injections | |
| 5 | 8 | Anti- | 0.24 mg/ | Every 3 days | 200 μL/mouse |
| | | CD71 | mouse/inj | with a total | |
| | | antibodies | | of 4 | |
| | | | | injections | |

The treatment takes place on days 5, 8, 11 and 14.

The results are presented in FIG. 2.

In group 1 (mice having received no treatment), a mouse was found dead on day 28, the other 7 were sacrificed between day 25 and day 32 after injection of the ARH-77 cells, due to the presence of pathological signs.

In group 2 (mice having received the irrelevant antibody at 0.048 mg/kg/injection), three mice died on day 35 and, 5 were sacrificed between days 29 and 96 after the injection of the ARH-77 cells and were sacrificed and finally 2 mice were sacrificed at the end of the study on day 106.

In group 3 (mice having received the irrelevant antibody at 0.24 mg/kg/injection), one mice died on day 28, 6 were sacrificed between day 29 and 32 after the injection of the ARH-77 cells and were sacrificed and finally 1 mouse was sacrificed at the end of the study on day 106.

In group 4 (mice having received the anti-CD71 antibody at 0.048 mg/kg/injection), one mouse was found dead on day 102, two were sacrificed on days 57 and 67 as they exhibited pathological signs and the other 5 survived the study and were sacrificed on day 106.

In group 5 (mice having received the anti-CD71 antibody at 0.24 mg/kg/injection) one mice was sacrificed on day 43 due to the presence of pathological signs and the other 7 survived the study and were sacrificed on day 106.

For the experiments, the animals were killed by cervical dislocation under anaesthesia with isoflurane if one of the following signs presented:
signs of suffering (cachexia, weakness, difficulty with movement and eating);
compound toxicity (convulsion)
a loss of 15% body weight over 3 consecutive days or 20% body weight in one day.
An autopsy by microscopy was carried out on all the animals which were sacrificed or died during these experiments. The autopsy relates to the heart, lungs, liver, spleen, kidneys, and intestine.

The isoflurane forene (Minerve, Bondoufle, France) was used to anaesthetize the mice before the injections of tumour cells by intravenous route and during the sacrifice of the animals by cervical dislocation. The viability, the clinical signs and behaviours were noted each day during the experiments. The body weight of each mouse was measured and noted twice a week.

3.2 Effect of the Anti-CD71 Antibody on the Survival of the Mice Bearing ARH-77 Tumour Cells 40 female mice with severe combined immunodeficiency (SCID) were first injected by intravenous route with a volume of 0.2 ml of the RPMI 1640 medium containing five million suspended ARH-77 tumour cells. The injection was carried out between 24 to 72 hours after whole-body exposure of the mice to gamma radiation (1.8 Gy, ⁶⁰Co, INRA, Dijon, France).

The day of injection of tumour cells is considered as day 0 (D0). The treatments with the anti-CD71 antibody and with the irrelevant antibody commence on day D−1. The treatment with Velcade® commences on day 4 and therefore takes place on days D4, D7, D11, D14, D18, D25, D28 and D32.

The 40 mice are separated into 5 groups. Table 4 below summarizes the treatment programme for each group.

TABLE 4

| | No. of | Substance | | Treatment | Injection |
|---|---|---|---|---|---|
| Group | mice | tested | Dose | programme | volume |
| 1 | 8 | Irrelevant | 1.2 mg/mouse/inj | Twice | 200 μL/mouse |
| | | antibody | | weekly for 6 | |
| | | | | weeks | |
| 2 | 8 | Anti- | 1.2 mg/mouse/inj | Twice | 200 μL/mouse |
| | | CD71 | | weekly for 6 | |
| | | antibody | | weeks | |
| 3 | 8 | Anti- | Anti-CD71 = 1.2 mg/ | Twice | Anti-CD71 = |
| | | CD71 | mouse/inj | weekly for 6 | 200 μL/mouse |
| | | antibody+ | Velcade ® = 0.5 mg/kg/ | weeks (anti- | Velcade = 10 ml/kg |
| | | Velcade ® | inj | CD71) + | |
| | | | | twice weekly | |
| | | | | for 4 weeks | |
| 4 | 8 | Velcade ® | 0.5 mg/kg/inj | twice weekly | Velcade = 10 ml/kg |
| | | | | for 4 weeks | |
| 5 | 8 | none | | | |

The injection volume of irrelevant antibody and anti-CD71 antibody is 200 μL/mouse/injection. The injection volume of Velcade® (10 ml/kg) is adjusted to the majority of the individual animals.

The mice surviving the experiment were sacrificed after 144 days following the injection of tumour cells. An autopsy was carried out on all the mice found dead or sacrificed during the experiment due to the presence of one of the pathological signs specified in the above section.

In group 1 (mice having received the irrelevant antibody), one mouse died on the 28^{th} day, 5 mice presented pathological signs between the 24^{th} day and the 56^{th} day after the injection of the ARH-77 cells and were sacrificed and finally 2 mice were sacrificed at the end of the study on D144.

In group 2 (mice having received the anti-CD71 antibody), a mice was found dead on the 42^{nd} day, one was sacrificed on the 98^{th} day as it presented pathological signs and the other 6 survived the study and were sacrificed on D144.

In group 3 (mice having received the anti-CD71 antibody and Velcade®, all the mice survived the study and were sacrificed on D144.

In group 4 (mice having received Velcade® only), 1 mouse died on the 27^{th} day, the other 7 were sacrificed due to the presence of pathological signs between day 24 and day 31. The autopsies revealed different signs (tumours on the bladder, the ovary, and kidney, small spleen or also inflammation on right axillary lymph nodes, and on left inguinal lymph nodes).

In group 5 (mice having received no treatment), the mice were sacrificed between day 24 and day 45 due to the presence of pathological signs.

The results are presented in FIG. 3.

Table 5 below compares the average survival of the mice in the 5 groups which died or were sacrificed before the end of the experiment due to the presence of the pathological signs specified above.

TABLE 5

| | Group 1 | Group 2 | Group 3 | Group 4 | Group 5 |
|---|---|---|---|---|---|
| The number of mice | 6 | 2 | 0 | 8 | 8 |
| that died before the | | | | | |
| end of the | | | | | |
| experiment | | | | | |
| The survival average | 41.5 | 70 | — | 28.4 | 29.6 |
| Standard deviation | 15.3 | 39.6 | — | 3.1 | 7 |
| The survival median | 55 | — | — | 29.5 | 29 |

Treatment with anti-CD71 antibody alone makes it possible to inhibit the development of a myeloma in the mice with severe combined immunodeficiency and prolong the survival of mice from 29.6 days, for the untreated mice, to more than 70 days for the mice treated by injection of anti-CD71 antibody.

The combination of anti-CD71 antibody with Velcade® has a synergistic effect on inhibiting myeloma development and prolonging the survival of affected mice, compared with the use of anti-CD71 antibody alone, or of Velcade® alone (FIG. 3). In all the mice having received this treatment, this combination succeeded in inhibiting myeloma development.

1. A method of preventing or treating myelomas, comprising administering to a subject in need thereof an effective amount of an anti-CD71 monoclonal antibody or a fragment of an abovementioned antibody capable of binding to the CD71 antigen, said antibody or said fragment of the abovementioned antibody comprising:
at least the variable region of a heavy chain comprising or constituted by the amino acid sequence SEQ ID NO: 1, and
at least the variable region of a light chain comprising or constituted by an amino acid sequence chosen from SEQ ID NO: 2,
SEQ ID NO: 11, SEQ ID NO: 13, SEQ ID NO: 15, SEQ ID NO: 16 or SEQ ID NO: 17.

2. The method according to claim 1, in which the constant regions of said light chains and heavy chains are the constant regions of a human antibody.

3. The method according to claim 1, in which
at least the constant region of a light chain comprises or is constituted by the amino acid sequence SEQ ID NO: 6 or SEQ ID NO: 14,
at least the constant region of a heavy chain comprises or is constituted by the amino acid sequence chosen from SEQ ID NO: 5 or SEQ ID NO: 9.

4. The method according to claim 1, in which said fragment is Fab, F(ab)′2, Fd, scFV, ScFv dimer, diabody, triabody or tetrabody.

5. The method according to claim 1, in which said antibody or said fragment of an antibody is coupled with a bioactive molecule chosen from:
radio-isotopes,
non-radioactive metals,
toxins chosen from ricin, abrin, diphtheria toxin,
nucleic acids chosen from the anti-sense RNAs,
cytotoxic agents chosen from mitomycin C, methotrexate, adriamycin,
enzymes such as the RNases,
biotin, avidin or streptavidin.

6. The method according to claim 1, in which the myeloma is chosen from stage I myeloma (low tumour mass), stage II myeloma (intermediate tumour mass), stage III myeloma (high tumour mass).

7. Product containing:
at least one anti-CD71 monoclonal antibody or of a fragment of an abovementioned antibody capable of binding to the CD71 antigen, said antibody or said fragment of the above-mentioned antibody comprising
at least the variable region of a heavy chain comprising or constituted by the amino acid sequence SEQ ID NO: 1, and
at least the variable region of a light chain comprising or constituted by an amino acid sequence chosen from SEQ ID NO: 2, SEQ ID NO: 11, SEQ ID NO: 13, SEQ ID NO: 15, SEQ ID NO: 16 or SEQ ID NO: 17,
an anticancer agent chosen from Velcade®, Melphalan®, Prednisone®.
as combination product, for use simultaneously, separately or spread over time in tumour therapy.

8. Product according to claim 7, in which the constant regions of said light chains and heavy chains are constant regions of a human antibody.

9. Product according to claim 8, in which:
at least the constant region of a light chain comprises or is constituted by the amino acid sequence SEQ ID NO: 6 or SEQ ID NO: 14,
at least the constant region of a heavy chain comprises or is constituted by the amino acid sequence chosen from SEQ ID NO: 5 or SEQ ID NO: 9.

10. Method for diagnosing myelomas in vitro, comprising:
(i) incubation of a cell sample taken from a patient suspected of having a myeloma, with an antibody anti-CD71 or a fragment of an abovementioned antibody capable of binding to the CD71 antigen, said antibody or said fragment of the abovementioned antibody comprising:
at least the variable region of a heavy chain comprising or constituted by the amino acid sequence SEQ ID NO: 1, and
at least the variable region of a light chain comprising or constituted by an amino acid sequence chosen from SEQ ID NO: 2, SEQ ID NO: 11, SEQ ID NO: 13, SEQ ID NO: 15, SEQ ID NO: 16 or SEQ ID NO: 17,
(ii) measurement of the quantity of CD71 antigens present on the surface of cells present in said sample,
(iii) comparison of the value obtained in the previous stage with a reference value established in a healthy cell sample.
